(19) United States
(12) Patent Application Publication
(10) Pub. No.: US 2013/0345546 A1
(43) Pub. Date: Dec. 26, 2013

(54) CT-MRI HYBRID APPARATUS WITH LARGER CT CORE-DIAMETER AND METHOD OF IMPLEMENTING THE SAME

(71) Applicant: GEORGES HOBEIKA, Miami, FL (US)

(72) Inventor: GEORGES HOBEIKA, Miami, FL (US)

(21) Appl. No.: 13/969,550

(22) Filed: Aug. 17, 2013

Related U.S. Application Data
(63) Continuation-in-part of application No. 13/244,531, filed on Sep. 25, 2011, now abandoned.

(51) Int. Cl. A61B 5/00; A61B 6/03

(52) U.S. Cl. CPC A61B 5/0035; A61B 6/032
USPC 600/411

(57) ABSTRACT

An apparatus and method for integrating computed tomography (CT) or computerized axial tomography (CAT) and magnetic resonance imaging (MRI) to provide high spatial-accurate resolution images. The apparatus includes a first section for obtaining a first set of images of a subject, the first section including a design of a CT machine with much larger dimension in height and width and very large opening or first diameter and redistribution of the electric gear peripherally, and a second section for obtaining a second set of images of the subject. More specifically, the CT machine design permits to position the CT x-ray tube, sensors and other electric and rotating gear substantially outside the magnetic field of the MRI machine, and the CT machine preferably includes three cameras or X-ray tubes at about 120 degree from each other for reduced scanning time.

### CROSS REFERENCE TO RELATED APPLICATIONS

This application is a continuation-in-part application and claims priority of U.S. application Ser. No. 13/244,531, filed on Sep. 25, 2011.

### BACKGROUND OF THE INVENTION

1. Field of the Invention

The present invention generally relates to an apparatus and method for non-invasively obtaining an image featuring information on internal human tissues, and more particularly to an apparatus and method for integrating computed tomography (CT) or computerized axial tomography (CAT) with magnetic resonance imaging (MRI). The present invention provides high spatial-accurate resolution images which feature tumor information and extension in relation to human internal tissues especially as a part of a radiotherapy treatment.

2. Discussion of the Related Art

A CT scan is an x-ray procedure that combines many x-ray images with the aid of a computer to generate cross-sectional views. In general, a CT scan costs less, takes shorter testing time, and is very good for imaging bone structures. However, CT images have a relatively low resolution in terms of soft tissue delineation and make it difficult to define with high precision disease infiltration limits, for example, extra-capsular extension in prostate cancer, or structures including glands, small organs, nerves and vessels. Nevertheless, CT images currently are the study of choice for the evaluation of bony anatomy and bilateral adrenal glands.

And, in radiotherapy, CT images form the sole basis for radiation dose computation algorithms. Routinely, radiation oncologists employ CT simulation, which involves three-dimensional image acquisition of the patient's body or region to be treated using CT scans, as a component of radiotherapy planning. More specifically, CT simulations are performed with the patient placed in the treatment position and form the basis for planning the radiotherapy treatment. From these images, normal structures as well as tumors are visually identified and delineated by oncologists. A treatment plan will then be generated respecting the parameters of radiation therapy, e.g., a particular radiation dose is given to the tumor, while minimizing the dose to the surrounding normal structures.

However, as technology for radiotherapy delivery advances, one of the goals is to achieve higher precision in dose delivery and conformity to the target or tumor, while sparing surrounding normal tissues. Therefore, although CT images alone form the sole basis for radiation dose computation algorithms, the use of MRI to accompany CT scans in radiotherapy planning, particularly to help in tumor and normal tissues delineation has become routine.

MRI makes use of the property of nuclear magnetic resonance to image nuclei of atoms inside a body. An MRI machine uses a magnetic field to align the magnetization of some atoms in the body, and radio frequency fields to systematically alter the alignment of this magnetization, thereby causing the nuclei to produce a signal detectable by the MRI scanner or detector. The MRI software then constructs an image of the scanned area of the body. Three-dimensional images offer gradients and provide excellent contrast between the different soft tissues of a body. As a result, MRI scan is far superior than CT scanning in anatomical details, it currently is the study of choice for delineation of most tumor and normal structures, and has become a routine companion imaging to CT simulation scans in radiotherapy planning to assist in delineating tumor from surrounding normal structures.

Currently, CT scanner and MRI scanner are located separately in different suites, and a patient is transported between suites to collect CT images and MRI images. This is because the MRI scanner maintains a magnetic field even when not in operation, and such a magnetic field interferes with the sensors of the CT scanner. In particular, the magnetic field generated by the MRI creates major interference to the electrical components of the CT scanner, e.g., the camera or rotating machinery. These electrical components are significantly altered under the magnetic influence and become dysfunctional in a very short period of time.

Subsequently, physics personnel skills and computer software programs are used to align or “fuse” CT simulation axial images with MRI axial images. The fused images are then used to help in identifying and contouring a tumor target and normal structures on the CT scan images, while comparing them head to head with the MRI images of the same axial plane. However, the current machines and method have major limited accuracy.

Oftentimes, and realistically, the CT and the MRI are performed in different body positions and the images generated from both studies are not exactly at the same level or in the same orientation. It should be mentioned that even minor differences in body positioning can generate a notable discrepancy between both sets of images and impair the quality of the “fusion”. Also if long interval time between the scans is permitted, physiologic internal organ motion might take place, accentuating the differences between the 2 sets of images, and rending the accuracy of the MR guided contouring process further reduced. Reduced accuracy defeats the purpose of including an MRI study as part of the simulation and treatment planning.

Therefore, what is needed is a hybrid device that can reduce and minimize differences in body positioning and time between sca ns. A device as such will increase the accuracy of tumor and normal tissues delineation, so that treatment planning can be improved. Such a design will allow a non compatible machines, CT and MRI, to be located in the same suite, and functioning more like a hybrid system, through the introduction of novel CT design.

### SUMMARY OF THE INVENTION

Accordingly, embodiments of the invention are directed to an apparatus and method for non-invasively obtaining an image featuring information on internal human tissues that substantially obviate one or more of the problems due to limitations and disadvantages of the related art.

An object of embodiments of the invention is to provide an apparatus and method for non-invasively obtaining an image featuring information on internal human tissues that integrate at least computed tomography (CT) or computerized axial tomography (CAT) and magnetic resonance imaging (MRI)

An object of embodiments of the invention is to provide an apparatus and method for non-invasively obtaining an image featuring information on internal human tissues that provide a single scanning board between a CT or CAT machine and a MRI machine, both located in the same suite, and where the CT is redesigned so that the CT or CAT sensors are located substantially outside of the significant impact of the magnetic field of the MRI machine, so that such hybrid system can exist and function.

Additional features and advantages of embodiments of the invention will be set forth in the description which follows, and in part will be apparent from the description, or may be learned by practice of embodiments of the invention. The objectives and other advantages of the embodiments of the invention will be realized and attained by the structure particularly pointed out in the written description and claims hereof as well as the appended drawings.

To achieve these and other advantages and in accordance with the purpose of embodiments of the invention, as embodied and broadly described, an apparatus and method for non-invasively obtaining an image featuring information on internal human tissues according to an embodiment of the present invention includes a first section for obtaining a first set of images of a subject using a first method, a second section for obtaining a second set of images of the subject using a second method, the second method being different from the first method, a newly designed first method to make it compatible with the second method, and a board for carrying the subject through the first and second sections, the board being movable between the first and second sections.

Another apparatus and method for non-invasively obtaining an image featuring information on internal human tissues according to an embodiment of the present invention includes a first section for obtaining a first set of images of a subject, the first section including one of a CT machine or a CAT machine having a first diameter, a second section for obtaining a second set of images of the subject, the second section including an MRI machine having a second diameter, and a board for carrying the subject through the first and second sections, the board being movable between the first and second sections, wherein the first diameter is larger than the second diameter.

According to an embodiment of the present invention, the first diameter of the CT machine or the CAT machine is about the maximum dimensions of an imaging suite, so that the CT or CAT components, including rotating elements, X-ray source, detectors, gear wheels and motors, are located substantially outside the core magnetic field of the MRI machine.

According to an embodiment of the present invention, the number of X-ray source is three, and the three X-ray sources are spaced about 120 degree apart from one another along the CT arc.

According to an embodiment of the present invention, the strength of the magnetic field by the MRI machine on the CT or CAT electric components is less than about ninety-five percent of the strongest strength of the magnetic field by the MRI machine.

It is to be understood that both the foregoing general description and the following detailed description are exemplary and explanatory and are intended to provide further explanation of embodiments of the invention as claimed.

### BRIEF DESCRIPTION OF THE DRAWINGS

The accompanying drawings, which are included to provide a further understanding of embodiments of the invention and are incorporated in and constitute a part of this specification, illustrate embodiments of the invention and together with the description serve to explain the principles of embodiments of the invention.

FIG. 1 is a side view of a CT machine according to the related art.

FIG. 2 is a perspective view of a hybrid image scanner according to an embodiment of the present invention.

FIG. 3 is a side view illustration of the magnetic field by the MRI machine of the hybrid image sensor shown in FIG. 2.

### DETAILED DESCRIPTION OF THE PREFERRED EMBODIMENTS

Reference will now be made in detail to the preferred embodiments of the invention, examples of which are illustrated in the accompanying drawings.

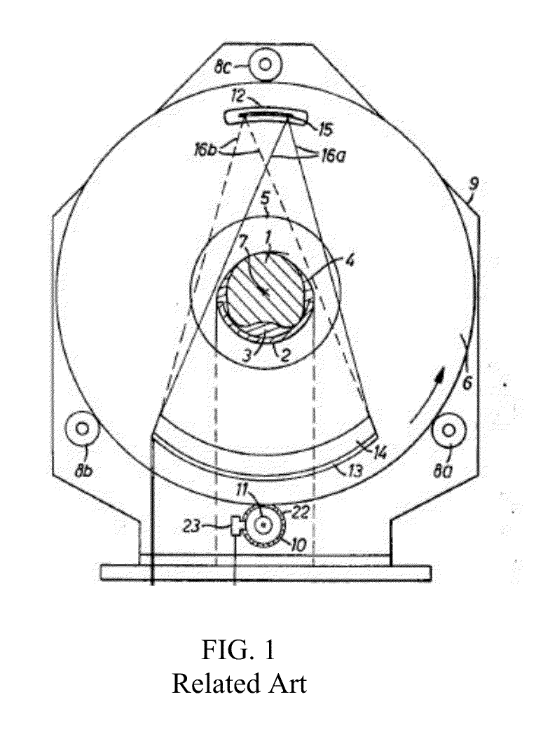

FIG. 1 is a side view of a CT machine according to the related art. As shown in FIG. 1, in a stand-alone CT machine according to the related art, a body 1 to be examined is positioned on a bed 2.

The bed 2 and the body 1 are inserted into an opening 5 in a rotatable member 6. In a preferred embodiment, the opening 5 preferably has a diameter of 70 to 100 cm. The rotatable member 6 is arranged to rotate about an axis 7, longitudinal of the body and perpendicular to the paper, intersecting the opening 5. The rotatable member 6 can be supported by three gear wheels 8a, 8b and 8c. The gear wheels 8 are in a main frame 9 of the CT machine. A further gear wheel 10 is driven by an electric motor 11, mounted on the main frame 9 and serves to provide the required rotary motion.

The rotatable member 6 also carries a source 12 of X-rays, a bank of detectors 13. The detectors 13 can be of any suitable type, for example scintillation crystals with associated photomultipliers or photodiodes.

The source 12 includes one elongated target/anode 15, and provides a fan shaped beam 16 of X-rays. The detectors are arranged to intercept the radiation of fan 16 for any position of the point of origin of the X-rays.

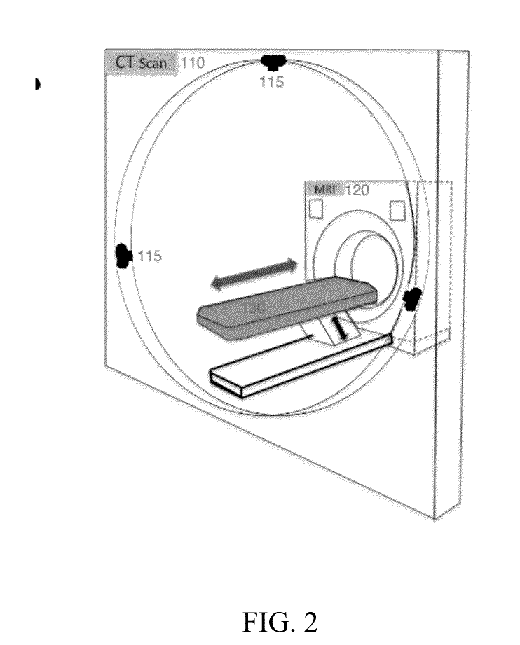

FIG. 2 is a perspective view of a hybrid image scanner according to an embodiment of the present invention. As shown in FIG. 2, a hybrid system 100 includes a first section 110 for obtaining images based on a first method and a second section 120 for obtaining images based on a second method. The first section 110 includes a computed tomography (CT) or computerized axial tomography (CAT). The second section 120 includes magnetic resonance imaging (MRI). Both of the first and second sections 110 and 120 are located in the same physical suite, and within about 1-2 meters from each other.

The first section 110 includes a new concept CT scanner designed and built to be compatible to be in the same suite as an MRI machine. In particular, the new concept CT scanner is very large in dimension, extending vertically and horizontally to the entire height and width of the imaging suite (about 4 to 5 meters) and has a large core diameter or opening 5 of almost same dimension (about 4 to 5 meters in diameter). The first section 110 has its rotating elements, X-ray source, detectors and other components of the CT scanner located outside of the effect of the magnetic field of the MRI machine. In particular, the gear wheels and the electric motor are redistributed into the four corners space of the main frame.

In a preferred embodiment, the first section 110 includes three X-ray sources, such as cameras or X-ray tube, located along the are of the CT rotating element. In particular, due to the large CT diameter, longer time is needed to complete a full rotation to acquire the image by the CT scanner. To reduce the rotational time and scan time, the first section 110 includes at least 3 cameras positioned spaced equally apart, e.g., at about 120 degrees from each other. The first section 110 rotates the three cameras simultaneously at the same speed, since they are mounted on the same rotating element. Therefore, the time to complete a full rotation can be reduced by a factor of 3, and the time to complete the CT image acquisition can be reduced by a factor of 3 for compensating at least partially the prolonged imaging time caused by a larger CT diameter.

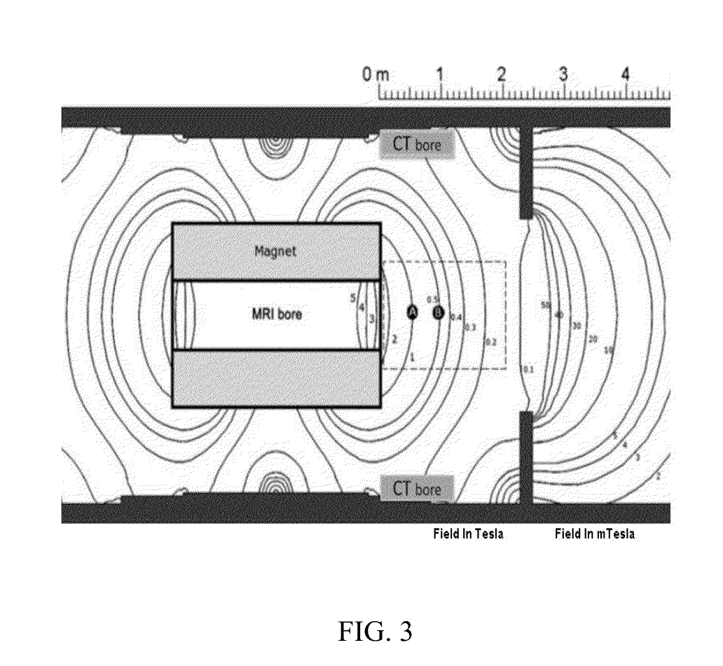

The core diameter of the CT scanner or opening is selected such that the strength of the magnetic field by the MRI machine on the CT or CAT electric components is less than about 95 percent of the strongest strength of the magnetic field by the MRI machine, in order to make any interference between the magnetic field and the CT scan negligible. FIG. 3 illustrates the magnetic field by a MRI machine (illustrated by the line) in Tesla on the left portion. As shown, the MRI bore generates substantially symmetrical bi-cylindrical-like magnetic field. According to an embodiment of the present invention, the core diameter of the CT scanner is built such that the components of the CT scanners are located outside the core strength of the MRI magnetic field. In other words, the components of the CT scanners are located where if there is any magnetic field by the MRI scanner, the strength of such magnetic field is very insignificant to produce any interference between both machines. In a preferred embodiment, the strength of the magnetic field by the MRI machine on the CT or CAT electric components is less than about ninety-five percent of the strongest strength of the magnetic field by the MRI machine.

Turning back to FIG. 2, the system 100 further includes an elongated scanning board 130 and a processor (not shown). A subject, such as a patient, is fixed onto the board 130 using immobilization devices such as masks or cradles. The height of the board 130 may be adjustable.

After the subject is fixed onto the board 130, the subject preferably first passes through the first section 110 and subsequently through the second section 120. The board 130 is movable and slidable along an axial through the first and second sections 110 and 120. While going through the first and second sections 110 and 120, the subject lies still with immobilization devices and undergoes a continuous multi-step scanning process. For example, a CT scan of the area of interest of the subject 140 can be followed by an MRI scan of that same area of interest.

In a preferred embodiment, the processor controls the board 130, as well as the scan speed or angle of the first and second sections 110 and 120.

A feature of the system 100 and the method of obtaining images using the system 100 is that the subject remains immobilized in the same exact position on the scanning board 130 during the entire image acquisition process. Thus, set-up errors or positioning uncertainties that could have been generated by separate scanning procedures are minimized or eliminated.

Additionally, the system 100 can obtain two different types of scans almost near simultaneous in time. Thus, any possible internal organ motion or change in configuration or size of certain tissues if both scans were more separated in time can be avoided. For example, as many organs, such as prostate gland, rectum, bladder, bowel, surgical bed or brain tissue, shift with time. The system 100 that obtain different types of scans immediately following one and another can provide even more accurate images for fusing or treatment planning.

The system 100 performs different types of scans to generate a highly accurate super-imposed sets of images that can be fused. For example, the imposed images or fused images, such as head to head in an axial plan, can be within sub-millimeter accuracy. Highly accurate images obtained by the system 100 subsequently provide a confident and precise delineation of structures on the CT images guided by the corresponding MRI images. Further, the images obtained by the system 100 provide a more realistic time representation of the actual anatomy displayed on the CT images.

It will be apparent to those skilled in the art that various modifications and variations can be made in the chassis structure of embodiments of the invention without departing from the spirit or scope of the invention. Thus, it is intended that embodiments of the invention cover the modifications and variations of this invention provided they come within the scope of the appended claims and their equivalents

The current design differ from a conventional PET/CT scan concept, which simply implies a CT scanner, a PET scanner and a common sliding board with both machine having similar design of a rotating element and a sliding board, and without any major incompatibility issues. Due the incompatibility of MRI and CT, there has been the absence of an MRI/CT scan machine as one system or one imaging machine, located in the same suite. Hence to make MRI and CT compatible in a same-suite system, the embodiments of this novel invention achieve these and other advantages and in accordance with the purpose of embodiments of the invention.

What is claimed:

1. A device, comprising:
a first section for obtaining a first set of images of a subject, the first section including one of a CT machine or a CAT machine having a first diameter;
a second section for obtaining a second set of images of the subject, the second section including an MRI machine having a second diameter; and
a board for carrying the subject through the first and second sections, the board being movable between the first and second sections,
wherein the first diameter is larger than the second diameter.

2. The device according to claim 1, wherein the first diameter of the CT machine or the CAT machine is about the maximum dimensions of an imaging suite, so that the CT or CAT components, including rotating elements, X-ray source, detectors, gear wheels and motors, are located substantially outside the core magnetic field of the MRI machine.

3. The device according to claim 2, wherein the number of X-ray source is three, and the three X-ray sources are spaced about 120 degree apart from one another along the CT arc.

4. The device according to claim 2, wherein the strength of the magnetic field by the MRI machine on the CT or CAT electric components is less than about ninety-five percent of the strongest strength of the magnetic field by the MRI machine.

5. The device according to claim 1, further comprising a processor for controlling how the board is moved through the first and second sections.

6. The device according to claim 1, further comprising an axial for supporting the board, wherein the board is slidable along the axial.

7. A method for medical procedure, comprising:
providing a movable board;
carrying a subject on the movable board through a first section of the procedure and a second section of the procedure;
moving the board into the first section and performing a first examination method on the subject while the subject is on the board;
moving the board into the second section and performing a second examination method on the subject while the subject is on the board,
wherein the first and second examination methods being different from one another; and combining results from the first and second examination methods for overlapping common information about the subject,
wherein the first section includes one of a CT machine or a CAT machine, and the second section includes an MRI machine, the diameter of the CT machine is larger than the diameter of the MRI machine.

8. The method according to claim 7, further comprising:
rotating three X-ray sources at about 120 degrees apart from one another along the CT arc.

9. The method according to claim 7, wherein the CT diameter is predetermined such that the CT or CAT electric components are located substantially outside the core magnetic field of the MRI machine.

10. The method according to claim 7, wherein the strength of the magnetic field by the MRI machine on the CT or CAT electric components is less than about ninety-five percent of the strongest strength of the magnetic field by the MRI machine.

11. The method according to claim 7, wherein the moving of the board into the first section and the moving of the board into the second section are controlled by using a processor.

12. The method according to claim 7, wherein the moving of the board into the first section and the moving of the board into the second section comprise the step of sliding the board along an axial located within the first and second sections.
